(19) United States
(12) Patent Application Publication
(10) Pub. No.: US 2009/0017161 A1
(43) Pub. Date: Jan. 15, 2009

(54) GENE ENCODING PROTEIN HAVING TREHALOSE SYNTHESIS-PROMOTING ACTIVITY AND USE THEREOF

(75) Inventors: Yoshihiro Nakao, Osaka (JP); Yukiko Kodama, Osaka (JP); Tomoko Shimonaga, Osaka (JP)

Correspondence Address: DRINKER BIDDLE & REATH (DC), 1500 K STREET, N.W., SUITE 1100, WASHINGTON, DC, 20005-1209, US

(73) Assignee: Suntory Limited, Osaka (JP)

(21) Appl. No.: 12/279,206

(22) PCT Filed: Feb. 1, 2007
(86) PCT No.: PCT/JP2007/052162

§ 371 Date: Aug. 13, 2008

(30) Foreign Application Priority Data
Feb. 28, 2006 (JP) 2006-054193

(51) Int. Cl. C12C 11/00; C12N 15/11; C07K 14/00; C12Q 1/68; C12N 15/00; C12N 1/19

(52) U.S. Cl. 426/15; 536/23.1; 530/350; 435/320.1; 435/254.2; 426/11; 426/16; 426/592; 435/6

(57) ABSTRACT

The present invention relates to a gene encoding a protein having a trehalose synthesis-promoting activity and use thereof, in particular, a yeast for practical use with superior resistance property to dryness and/or low-temperature storage, alcoholic beverages produced with said yeast, and a method for producing said beverages. More particularly, the present invention relates to a yeast, whose resistance property to dryness and/or resistance property to low-temperature storage is enhanced by amplifying expression level of TSL1 gene encoding a protein Ts11p having a trehalose synthesis-promoting activity in brewer's yeast, especially non-ScTSL1 gene specific to a lager brewing yeast and to a method for producing alcoholic beverages with said yeast, etc.

### TECHNICAL FIELD

The present invention relates to a gene encoding a protein having a trehalose synthesis-promoting activity and use thereof, in particular, a yeast for practical use with superior resistance property to dryness and/or resistance property to low-temperature storage, alcoholic beverages produced with said yeast, and a method for producing said beverages. More particularly, the present invention relates to a yeast, whose resistance property to dryness and/or resistance property to low-temperature storage is enhanced by amplifying expression level of TSL1 gene encoding a protein Ts11p having a trehalose synthesis-promoting activity in brewer's yeast, especially non-ScTSL1 gene specific to a lager brewing yeast and to a method for producing alcoholic beverages with said yeast, etc. Further, the yeast of the present invention is useful as a baker's yeast or an industrial yeast as well.

### BACKGROUND ART

Beer brewing is characterized by a process recovering yeasts after fermentation and using the recovered yeasts at the subsequent fermentation, which is called “Renjo”. The yeasts are stored in the presence of ethanol in a tank whose temperature is kept at approximately 0 to 3° C. When the yeasts die during the storage, not only the next fermentation process is interfered, but also constituents of the yeast cells released by cell lysis may impart unfavorable taste to product. Therefore, it is very important for allowing some variance to design production process and for stable production of quality products to use yeasts with superior resistant property to low-temperature storage.

“Renjo” may be terminated at a certain times of fermentation is carried out. The number of times of “Renjo” may vary according to fermentation conditions or properties of yeasts used in the process. A process to develop yeasts for fermentation freshly is called propagation. Yeasts are subcultured several times enlarging scales of culture successively during the propagation process. Because propagation process requires from several days to several weeks, it brings great advantages in production efficiency if term of the process is shortened or yeast cells which are large-scale pre-cultured are able to be stored stably for extended period of time at low temperature or under dry condition.

Concerning a method for producing dry yeast maintaining high viable cell ratio, improvement of drying equipment, or improvement of manufacturing conditions such as temperature or addition of emulsifiers, etc. have been made. For example, L-drying method is not practical to be used at industrial production scale because, though it can maintain extremely high viable cell ratio, but at the same time it takes a lot of time and cost.

Regarding low-temperature resistance of yeast, some experiments designed to improve refrigeration-resistant property mainly of baker's yeast were reported. This is because Saccharomyces cerevisiae, which is a baker's yeast, has poor low-temperature storage property in comparison with brewer's yeast for beer or sake, which can ferment at low temperature. For example, baker's yeasts having refrigeration-resistant property and drying-resistant property were found out mainly by screening methods in Japanese Patent Application Laid-open No. H11-155559 and Japanese Patent Application Laid-open No. 2003-304864. Further, regarding examples utilizing genetic engineering techniques, trehalose highly accumulating strains by disruption of NTH1, which is a trehalase gene, is reported in Japanese Patent Application Laid-open No. H10-117771 and a strain highly accumulating specific amino acids such as arginine by disruption of CAR1, which is an arginase gene, is reported in Japanese Patent Application Laid-open No. 2001-238665.

### DISCLOSURE OF INVENTION

Under the above situations, there has been a need to make high-efficiency production of alcoholic beverages or useful materials possible by using a gene encoding a protein responsible for drying and/or low-temperature storage-resistant property of brewery yeast and said protein.

The present inventors made extensive studies to solve the above problems and as a result, succeeded in identifying and isolating a gene encoding a protein having a trehalose synthesis-promoting activity from beer yeast. Moreover, the present inventors produced transformed yeast in which the obtained gene was expressed to verify that drying-resistant property and/or low-temperature storage-resistant property can be actually improved, thereby completing the present invention.

Thus, the present invention relates to a gene encoding a protein having a trehalose synthesis-promoting activity of brewery yeast, to a protein encoded by said gene, to a transformed yeast in which the expression of said gene is controlled, to a method for enhancing drying-resistant property and/or low-temperature storage-resistant proper of yeast using a yeast in which the expression of said gene is controlled, or the like. More specifically, the present invention provides the following polynucleotides, a vector comprising said polynucleotide, a transformed yeast introduced with said vector, a method for producing alcoholic beverages by using said transformed yeast, and the like.

(1) A polynucleotide selected from the group consisting of:

(a) a polynucleotide comprising a polynucleotide consisting of the nucleotide sequence of SEQ ID NO: 1;

(b) a polynucleotide comprising a polynucleotide encoding a protein consisting of the amino acid sequence of SEQ ID NO:2;

(c) a polynucleotide comprising a polynucleotide encoding a protein consisting of the amino acid sequence of SEQ ID NO:2 in which one or more amino acids thereof are deleted, substituted, inserted and/or added, and having a trehalose synthesis-promoting activity;

(d) a polynucleotide comprising a polynucleotide encoding a protein having an amino acid sequence having 60% or higher identity with the amino acid sequence of SEQ ID NO:2, and said protein having a trehalose synthesis-promoting activity;

(e) a polynucleotide comprising a polynucleotide which hybridizes to a polynucleotide consisting of a nucleotide sequence complementary to the nucleotide sequence of SEQ ID NO: 1 under stringent conditions, and which encodes a protein having a trehalose synthesis-promoting activity; and

(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO:2 under stringent conditions, and which encodes a protein having a trehalose synthesis-promoting activity.

(2) The polynucleotide according to (1) above selected from the group consisting of:

(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has a trehalose synthesis-promoting activity;

(h) a polynucleotide comprising a polynucleotide encoding a protein having 90% or higher identity with the amino acid sequence of SEQ ID NO: 2, and having a trehalose synthesis-promoting activity; and

(i) a polynucleotide comprising a polynucleotide which hybridizes to a polynucleotide consisting of a nucleotide sequence of SEQ ID NO: 1 or which hybridizes to a polynucleotide consisting of a nucleotide sequence complementary to the nucleotide sequence of SEQ ID NO: 1, under high, stringent conditions, which encodes a protein having a trehalose synthesis-promoting activity.

(3) The polynucleotide according to (1) above comprising a polynucleotide consisting of the nucleotide sequence of SEQ ID NO: 1.

(4) The polynucleotide according to (1) above comprising a polynucleotide encoding a protein consisting of the amino acid sequence of SEQ ID NO: 2.

(5) The polynucleotide according to any one of (1) to (4) above, wherein the polynucleotide is DNA.

(6) A protein encoded by the polynucleotide according to any one of (1) to (5) above.

(7) A vector containing the polynucleotide according to any one of (1) to (5) above.

(7a) The vector of (7) above, which comprises the expression cassette comprising the following components:

(x) a promoter that can be transcribed in a yeast cell,

(y) any of the polynucleotides described in (1) to (5) above linked to the promoter in a sense or antisense direction; and

(z) a signal that can function in a yeast with respect to transcription termination and polyadenylation of a RNA molecule.

(7b) The vector of (7) above, which comprises the expression cassette comprising the following components:

(x) a promoter that can be transcribed in a yeast cell;

(y) any of the polynucleotides described in (1) to (5) above linked to the promoter in a sense direction; and

(z) a signal that can function in a yeast with respect to transcription termination and polyadenylation of a RNA molecule.

(8) A yeast into which the vector according to any one of (7) to (7b) above has been introduced.

(9) The yeast (yeast for practical use) according to (8) above, wherein drying-resistant property is increased. The “yeast for practical use” means that a yeast which possesses practical value such as brewer's (brewery) yeast, baker's yeast or industrial yeast, etc.

(10) The yeast according to (8) above, wherein low-temperature storage-resistant property is increased.

(11) The yeast according to (9) above, wherein the drying-resistant property is increased by increasing an expression level of the protein of (6) above.

(12) The yeast according to (10) above, wherein the low-temperature storage-resistant property is increased by increasing an expression level of the protein of (6) above.

(12a) The yeast according to any one of (9) to (12) above, wherein the yeast is a brewery yeast.

(13) A method for producing an alcoholic beverage by using the yeast according to any one of (8) to (12a) above.

(14) The method according to (13) above, wherein the brewed alcoholic beverage is a malt beverage.

(15) The method according to (13) above, wherein the brewed alcoholic beverage is wine.

(16) An alcoholic beverage produced by the method according to any one of (13) to (15) above.

(17) A method for assessing a test yeast for its drying-resistant property and/or low-temperature storage-resistant property, comprising using a primer or probe designed based on the nucleotide sequence of a gene having the nucleotide sequence of SEQ ID NO: 1 and encoding a protein having a trehalose synthesis-promoting activity.

(17a) A method for selecting a yeast having an increased drying-resistant property and/or low-temperature storage-resistant property by using the method described in (17) above.

(17b) A method for producing an alcoholic beverage (for example, beer or alcohol for industrial use, etc.) by using the yeast selected with the method described in (17a) above.

(17c) A method for producing an useful materials (for example, protein) by using the yeast selected with the method described in (17a) above.

(18) A method for assessing a test yeast for its drying-resistant property and/or low-temperature storage-resistant property, comprising: culturing the test yeast; and measuring the expression level of the gene having the nucleotide sequence of SEQ ID NO: 1 and encoding a protein having a trehalose synthesis-promoting activity.

(18a) A method for selecting a yeast having a high drying-resistant property and/or low-temperature storage-resistant property, which comprises assessing a test yeast by the method described in (18) above and selecting a yeast having a high expression level of gene encoding a protein having a trehalose synthesis-promoting activity.

(18b) A method for producing an alcoholic beverage (for example, beer) by using the yeast selected with the method described in (18a) above.

(18c) A method for producing an useful material (for example, protein) by using the yeast selected with the method described in (18a) above.

(19) A method for selecting a yeast, comprising: culturing test yeasts; quantifying the protein of (6) above or measuring the expression level of the gene having the nucleotide sequence of SEQ ID NO: 1 and encoding a protein having a trehalose synthesis-promoting activity; and selecting a test yeast having an amount of the protein or the gene expression level according to favorable drying-resistant property and/or low-temperature storage-resistant property.

(20) The method for selecting a yeast according to (19) above, comprising: culturing a reference yeast and test yeasts; measuring for each yeast the expression level of the gene having the nucleotide sequence of SEQ ID NO: 1 and encoding a protein having a trehalose synthesis-promoting activity; and selecting a test yeast having the gene expression higher than that in the reference yeast.

(21) The method for selecting a yeast according to (19) above, comprising: culturing a reference yeast and test yeasts; quantifying the protein according to (6) above in each yeast; and selecting a test yeast having a larger amount of the protein than that in the reference yeast.

(22) A method for producing an alcoholic beverage comprising: conducting fermentation using the yeast according to any one of (8) to (12a) above or a yeast selected by the methods according to any one of (19) to (21) above.

The transformed yeast of the present invention is able to keep high viable cell ratio during dry storage or low-temperature storage. Therefore, when it is used for brewing and so on, painfulness of conserving yeast can be eliminated. Further, it is expected to contribute to quality stabilization. Moreover, dry yeast is suitable for long-storage, and it is very advantageous to distribution or transportation due to its reduced weight. It is also useful as microorganisms for industrial application such as industrial alcohol production or production of useful proteins. The yeast of the present invention also useful as an industrial yeast as well.

### BRIEF DESCRIPTION OF DRAWINGS

FIG. 1 shows the cell growth with time upon beer fermentation test. The horizontal axis represents fermentation time while the vertical axis represents optical density at 660 m (OD660).

FIG. 2 shows the extract (sugar) consumption with time upon beer fermentation test. The horizontal axis represents fermentation time while the vertical axis represents apparent extract concentration (w/w %).

FIG. 3 shows the expression profile of non-ScTSL1 gene in yeasts upon beer fermentation test. The horizontal axis represents fermentation time while the vertical axis represents the intensity of detected signal.

FIG. 4 shows the result of drying-resistant property test of parent strain and non-ScTSL1 highly expressed strain.

### BEST MODES FOR CARRYING OUT THE INVENTION

The present inventors isolated and identified non-ScTSL1 gene encoding a protein having a trehalose synthesis-promoting activity of brewery yeast based on the lager brewing yeast genome information mapped according to the method disclosed in Japanese Patent Application Laid-Open No. 2004-283169. The nucleotide sequence of the gene is represented by SEQ ID NO: 1. Further, an amino acid sequence of a protein encoded by the gene is represented by SEQ ID NO: 2.

### 1. Polynucleotide of the Invention

First of all, the present invention provides (a) a polynucleotide comprising a polynucleotide of the nucleotide sequence of SEQ ID NO:1; and (b) a polynucleotide comprising a polynucleotide encoding a protein of the amino acid sequence of SEQ ID NO:2. The polynucleotide can be DNA or RNA.

The target polynucleotide of the present invention is not limited to the polynucleotide encoding a protein having a trehalose synthesis-promoting activity described above and may include other polynucleotides encoding proteins having equivalent functions to said protein. Proteins with equivalent functions include, for example, (c) a protein of an amino acid sequence of SEQ ID NO:2 with one or more amino acids thereof being deleted, substituted, inserted and/or added and having a trehalose synthesis-promoting activity.

Such proteins include a protein consisting of an amino acid sequence of SEQ ID NO:2 with, for example, 1 to 100, 1 to 90, 1 to 80, 1 to 70, 1 to 60, 1 to 50, 1 to 40, 1 to 39, 1 to 38, 1 to 37, 1 to 36, 1 to 35, 1 to 34, 1 to 33, 1 to 32, 1 to 31, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6 (1 to several amino acids), 1 to 5, 1 to 4, 1 to 3, 1 to 2, or 1 amino acid residues thereof being deleted, substituted, inserted and/or added and having a trehalose synthesis-promoting activity. In general, the number of deletions, substitutions, insertions, and/or additions is preferably smaller. In addition, such proteins include (d) a protein having an amino acid sequence with about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher, or 99.9% or higher identity with the amino acid sequence of SEQ ID NO:2, and having a trehalose synthesis-promoting activity. In general, the percentage identity is preferably higher.

Trehalose synthesis-promoting activity may be evaluated by measuring trehalose synthase activity and/or trehalose level, for example, by a method described in Walter et al., J. Biol. Chem., 273, 33311-33319 (1993).

Furthermore, the present invention also contemplates (e) a polynucleotide comprising a polynucleotide which hybridizes to a polynucleotide consisting of a nucleotide sequence complementary to the nucleotide sequence of SEQ ID NO: 1 under stringent conditions and which encodes a protein having a trehalose synthesis-promoting activity; and (f) a polynucleotide comprising a polynucleotide which hybridizes to a polynucleotide complementary to a nucleotide sequence of encoding a protein of SEQ ID NO:2 under stringent conditions, and which encodes a protein having a trehalose synthesis-promoting activity.

Herein, “a polynucleotide that hybridizes under stringent conditions” refers to nucleotide sequence, such as a DNA, obtained by a colony hybridization technique, a plaque hybridization technique, a southern hybridization technique or the like using all or part of polynucleotide of a nucleotide sequence complementary to the nucleotide sequence of SEQ ID NO: 1 or polynucleotide encoding the amino acid sequence of SEQ ID NO:2 as a probe. The hybridization method may be a method described, for example, in MOLECULAR CLONING 3rd Ed., CURRENT PROTOCOLS IN MOLECULAR BIOLOGY, John Wiley & Sons 1987-1997, and so on.

The term “stringent conditions” as used herein may be any of low stringency conditions, moderate stringency conditions or high stringency conditions. “Low stringency conditions” are, for example, 5×SSC, 5×Denhardt's solution, 0.5% SDS, 50% formamide at 32° C. “Moderate stringency conditions” are, for example, 5×SSC, 5×Denhardt's solution, 0.5% SDS, 50% formamide at 42° C. “High stringency conditions” are, for example, 5×SSC, 5×Denhardt's solution, 0.5% SDS, 50% formamide at 50° C. Under these conditions, a polynucleotide, such as a DNA, with higher homology is expected to be obtained efficiently at higher temperature, although multiple factors are involved in hybridization stringency including temperature, probe concentration, probe length, ionic strength, time, salt concentration and others, and one skilled in the art may appropriately select these factors to realize similar stringency.

When a commercially available kit is used for hybridization, for example, Alkphos Direct Labeling Reagents (Amersham Pharmacia) may be used. In this case, according to the attached protocol, after incubation with a labeled probe overnight, the membrane is washed with a primary wash buffer containing 0.1% (w/v) SDS at 55° C., thereby detecting hybridized polynucleotide, such as DNA.

Other polynucleotides that can be hybridized include polynucleotides having about 60% or higher, about 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identity to polynucleotide encoding the amino acid sequence of SEQ ID NO:2 as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Natl. Acad. Sci. USA, 90: 5873, 1993). Programs called BLASTN and BLASTX based on BLAST algorithm have been developed (Altschul S F et al., J. Mol. Biol. 215: 403, 1990). When a nucleotide sequence is sequenced using BLASTN, the parameters are, for example, score=100 and word length=12. When an amino acid sequence is sequenced using BLASTX the parameters are, for example, score=50 and word length=3. When BLAST and Gapped BLAST programs are used, default parameters for each of the programs are employed.

### 2. Protein of the Present Invention

The present invention also provides proteins encoded by any of the polynucleotides (a) to (i) above. A preferred protein of the present invention comprises an amino acid sequence of SEQ ID NO:2 with one or several amino acids thereof being deleted, substituted, inserted and/or added, and having a trehalose synthesis-promoting activity.

Such protein includes those having an amino acid sequence of SEQ ID NO:2 with amino acid residues thereof of the number mentioned above being deleted, substituted, inserted and/or added and having a trehalose synthesis-promoting activity. In addition, such protein includes those having homology as described above with the amino acid sequence of SEQ ID NO:2 and having a trehalose synthesis-promoting activity.

Such proteins may be obtained by employing site-directed mutation described, for example, in MOLECULAR CLONING 3rd Ed., CURRENT PROTOCOLS IN MOLECULAR BIOLOGY, Nuc. Acids. Res., 10: 6487 (1982), Proc. Natl. Acad. Sci. USA 79: 6409 (1982), Genie 34: 315 (1985), Nuc. Acids. Res., 13: 4431 (1985), Proc. Natl. Acad. Sci. USA 82: 488 (1985).

Deletion, substitution, insertion and/or addition of one or more amino acid residues in an amino acid sequence of the protein of the invention means that one or more amino acid residues are deleted, substituted, inserted and/or added at any one or more positions in the same amino acid sequence. Two or more types of deletion, substitution, insertion and/or addition may occur concurrently.

Hereinafter, examples of mutually substitutable amino acid residues are enumerated. Amino acid residues in the same group are mutually substitutable. The groups are provided below.

Group A: leucine, isoleucine, norleucine, valine, norvaline, alanine, 2-aminobutanoic acid, methionine, o-methylserine, t-butylglycine, t-butylalanine, cyclohexylalanine; Group B: asparatic acid, glutamic acid, isoasparatic acid, isoglutamic acid, 2-aminoadipic acid, 2-aminosuberic acid; Group C: asparagine, glutamine; Group D: lysine, arginine, ornithine, 2,4-diaminobutanoic acid, 2,3-diaminoproprionic acid; Group E: proline, 3-hydroxyproline, 4-hydroxyproline; Group F: serine, threonine, homoserine; and Group G: phenylalanine, tyrosine.

The protein of the present invention may also be produced by chemical synthesis methods such as Fmoc method (fluorenylmethyloxycarbonyl method) and tBoc method (t-butyloxycarbonyl method). In addition, peptide synthesizers available from, for example, Advanced ChemTech, PerkinElmer, Pharmacia, Protein Technology Instrument, Synthecell-Vega, PerSeptive, Shimazu Corp. can also be used for chemical synthesis.

3. Vector of the Invention and Yeast Transformed with the Vector

The present invention then provides a vector comprising the polynucleotide described above. The vector of the present invention is directed to a vector including any of the polynucleotides described in (a) to (i) above or any of the polynucleotides described in (j) to (m) above. Generally, the vector of the present invention comprises an expression cassette including as components (x) a promoter that can transcribe in a yeast cell; (y) a polynucleotide described in any of (a) to (i) above that is linked to the promoter in sense or antisense direction; and (z) a signal that functions in the yeast with respect to transcription termination and polyadenylation of RNA molecule. Further, in order to highly express the protein of the invention, these polynucleotides are preferably introduced in the sense direction to the promoter to promote expression of the polynucleotide (DNA) described in any of (a) to (i) above.

A vector introduced in the yeast may be any of a multicopy type (YEp type), a single copy type (YCp type), or a chromosome integration type (YIp type). For example, YEp24 (J. R Broach et al., EXPERIMENTAL MANIPULATION OF GENE EXPRESSION, Academic Press, New York 83, 1983) is known as a YEp type vector, YCp50 (M. D. Rose et al., Gene 60: 237, 1987) is known as a YCp type vector, and YIp5 (K. Struhl et al., Proc. Natl. Acad. Sci. USA, 76: 1035, 1979) is known as a YIp type vector, all of which are readily available.

Promoters/terminators for adjusting gene expression in yeast may be in any combination as long as they function in the yeast for practical use and they are not influenced by constituents in fermentation broth. For example, a promoter of glyceraldehydes 3-phosphate dehydrogenase gene (TDH3), or a promoter of 3-phosphoglycerate kinase gene (PGK1) may be used. These genes have previously been cloned, described in detail, for example, in M. F. Tuite et al., EMBO J., 1, 603 (1982), and are readily available by known methods.

Since an auxotrophy marker cannot be used as a selective marker upon transformation for a yeast for practical use, for example, a geneticin-resistant gene (G418r), a copper-resistant gene (CUP1) (Marin et al., Proc. Natl. Acad. Sci. USA, 81, 337 1984) or a cerulenin-resistant gene (fas2m, PDR4) (Junji Inokoshi et al., Biochemistry, 64, 660, 1992; and Hussain et al., Gene, 101: 149, 1991, respectively) may be used.

A vector constructed as described above is introduced into a host yeast. Examples of the host yeast include any yeast (yeast for practical use) that can be used for brewing, for example, brewery yeasts for beer, wine and sake, baker's yeast, yeast for producing industrial alcohol or yeast for producing useful proteins and so on. Specifically, yeasts such as genus Saccharomyces may be used. According to the present invention, a lager brewing yeast, for example, Saccharomyces pastorianus W34/70, etc., Saccharomyces carlsbergensis NCYC453 or NCYC456, etc., or Saccharomyces cerevisiae NBRC1951, NBRC1952, NBRC1953 or NBRC1954, etc., may be used. In addition, whisky yeasts such as Saccharomyces cerevisiae NCYC90, wine yeasts such as wine yeasts #1, 3 and 4 from the Brewing Society of Japan, and sake yeasts such as sake yeast #7 and 9 from the Brewing Society of Japan, baker's yeast such as NBRC0555, NBRC1346 or NBRC2043, etc., may also be used but not limited thereto. In the present invention, lager brewing yeasts such as Saccharomyces pastorianus may be used preferably.

A yeast transformation method may be a generally used known method. For example, methods that can be used include but not limited to an electroporation method (Meth. Enzym., 194: 182 (1990)), a spheroplast method (Proc. Natl. Acad. Sci. USA, 75: 1929 (1978)), a lithium acetate method (J. Bacteriology, 153: 163 (1983)), and methods described in Proc. Natl. Acad. Sci. USA, 75: 1929 (1978), METHODS IN YEAST GENETICS, 2000 Edition: A Cold Spring Harbor Laboratory Course Manual.

More specifically, a host yeast is cultured in a standard yeast nutrition medium (e.g., YEPD medium (Genetic Engineering. Vol. 1, Plenum Press, New York, 117 (1979)), etc.) such that OD600 nm will be 1 to 6. This culture yeast is collected by centrifugation, washed and pre-treated with alkali metal ion, preferably lithium ion at a concentration of about 1 to 2 M. After the cell is left to stand at about 30° C. for about 60 minutes, it is left to stand with DNA to be introduced (about 1 to 20 μg) at about 30° C. for about another 60 minutes. Polyethyleneglycol, preferably about 4,000 Dalton of polyethyleneglycol, is added to a final concentration of about 20% to 50%. After leaving at about 30° C. for about 30 minutes, the cell is heated at about 42° C. for about 5 minutes. Preferably, this cell suspension is washed with a standard yeast nutrition medium, added to a predetermined amount of fresh standard yeast nutrition medium and left to stand at about 30° C. for about 60 minutes. Thereafter, it is seeded to a standard agar medium containing an antibiotic or the like as a selective marker to obtain a transformant.

Other general cloning techniques may be found, for example, in MOLECULAR CLONING 3rd Ed., and METHODS IN YEAST GENETICS, A LABORATORY MANUAL (Cold Spring Harbor Laboratory Press, Cold Spring Harbor, N.Y.).

### 4. Method of Producing Alcoholic Beverages According to the Present Invention and Alcoholic Beverages Produced by the Method

A yeast having a superior drying-resistant property and/or low-temperature storage-resistant property can be obtained by introducing the vector of the present invention described above to a yeast. Further, a yeast having a superior drying-resistant property and/or low-temperature storage-resistant property can be obtained by selecting a yeast by the yeast assessment method of the present invention described below. The target use of yeasts obtained in the present invention include, for example, but not limited to, brewing alcoholic beverages such as beer, wine, whisky, sake and the like, baking bread, manufacturing useful materials such as industrial alcohol production and production of useful proteins.

In order to produce these products, a known technique can be used except that a yeast for practical use obtained according to the present invention is used in the place of a parent strain. Since starting materials, manufacturing equipment, manufacturing control and the like may be the same as the conventional ones; it can be performed without increasing cost.

### 5. Yeast Assessment Method of the Invention

The present invention relates to a method for assessing a test yeast for its drying-resistant property and/or low-temperature storage-resistant property by using a primer or a probe designed based on a nucleotide sequence of a gene having the nucleotide sequence of SEQ ID NO: 1 and encoding a protein having a trehalose synthesis-promoting activity. General technique for such assessment method is known and is described in, for example, WO01/040541, Japanese Laid-Open Patent Application No. H8-205900 or the like. This assessment method is described in below.

First, genome of a test yeast is prepared. For this preparation, any known method such as Hereford method or potassium acetate method may be used (e.g., METHODS IN YEAST GENETICS, Cold Spring Harbor Laboratory Press, 130 (1990)). Using a primer or a probe designed based on a nucleotide sequence (preferably, ORF sequence) of the gene encoding a protein having a trehalose synthesis-promoting activity, the existence of the gene or a sequence specific to the gene is determined in the test yeast genome obtained. The primer or the probe may be designed according to a known technique.

Detection of the gene or the specific sequence may be carried out by employing a known technique. For example, a polynucleotide including part or all of the specific sequence or a polynucleotide including a nucleotide sequence complementary to said nucleotide sequence is used as one primer, while a polynucleotide including part or all of the sequence upstream or downstream from this sequence or a polynucleotide including a nucleotide sequence complementary to said nucleotide sequence, is used as another primer to amplify a nucleic acid of the yeast by a PCR method, thereby determining the existence of amplified products and molecular weight of the amplified products. The number of bases of polynucleotide used for a primer is generally 10 base pairs (bp) or more, and preferably 15 to 25 bp. In general, the number of bases between the primers is suitably 300 to 2000 bp.

The reaction conditions for PCR are not particularly limited but may be, for example, a denaturation temperature of 90 to 95° C., an annealing temperature of 40 to 60° C., an elongation temperature of 60 to 75° C., and the number of cycle of 10 or more. The resulting reaction product may be separated, for example, by electrophoresis using agarose gel to determine the molecular weight of the amplified product. This method allows prediction and assessment of the drying-resistant property and/or low-temperature storage-resistant property of yeast as determined by whether the molecular weight of the amplified product is a size that contains the DNA molecule of the specific part. In addition, by analyzing the nucleotide sequence of the amplified product, the property may be predicted and/or assessed more precisely.

Moreover, in the present invention, a test yeast is cultured to measure an expression level of the gene encoding a protein having a trehalose synthesis-promoting activity having the nucleotide sequence of SEQ ID NO: 1 to assess the test yeast for its drying-resistant property and/or low-temperature storage-resistant property. Measurement of expression level of the gene encoding a protein having a trehalose synthesis-promoting activity can be performed by culturing test yeast and then quantifying mRNA or a protein resulting from the gene. The quantification of mRNA or protein may be carried out by employing a known technique. For example, mRNA may be quantified, by Northern hybridization or quantitative RT-PCR, while protein may be quantified, for example, by Western blotting (CURRENT PROTOCOLS N MOLECULAR BIOLOGY, John Wiley & Sons 1994-2003).

Furthermore, test yeasts are cultured and expression levels of the gene encoding a protein having a trehalose synthesis-promoting activity having the nucleotide sequence of SEQ ID NO:1 are measured to select a test yeast with the gene expression level according to the target trehalose-producing ability, thereby a yeast favorable for brewing desired alcoholic beverages can be selected. In addition, a reference yeast and a test yeast may be cultured so as to measure and compare the expression level of the gene in each of the yeasts, thereby a favorable test yeast can be selected. More specifically, for example, a reference yeast and one or more test yeasts are cultured and an expression level of the gene encoding a protein having a trehalose synthesis-promoting activity having the nucleotide sequence of SEQ ID NO: 1 is measured in each yeast. By selecting a test yeast with the gene expressed higher than that in the reference yeast, a yeast suitable for brewing desired alcoholic beverages or production of useful materials can be selected.

Alternatively, test yeasts are cultured and a yeast with a high trehalose-producing ability is selected, thereby a yeast suitable for brewing desired alcoholic beverages or production of useful materials can be selected.

In these cases, the test yeasts or the reference yeast may be, for example, a yeast introduced with the vector of the invention, an artificially mutated yeast or a naturally mutated yeast. The trehalose synthesis-promoting activity can be evaluated by measuring trehalose synthesis-promoting activity and/or trehalose level by, for example, a method described in Walter et al., J. Biol. Chem., 273, 33311-33319 (1993). The mutation treatment may employ any methods including, for example, physical methods such as ultraviolet irradiation and radiation irradiation, and chemical methods associated with treatments with drugs such as EMS (methylmethane sulphonate) and N-methyl-N-nitrosoguanidine (see, e.g., Yasuji Oshima Ed., BIOCHEMISTRY EXPERIMENTS vol. 39, Yeast Molecular Genetic Experiments, pp. 67-75, JSSP).

In addition, examples of yeasts used as the reference yeast or the test yeasts include any yeasts (yeasts for practical use), for example, brewery yeasts for beer, wine, sake and the like or baker's yeast, yeast for producing industrial alcohol or yeast for producing useful proteins, etc. More specifically, yeasts such as genus Saccharomyces may be used (e.g., S. pastorianus, S. cerevisiae, and S. carlsbergensis). According to the present invention, a lager brewing yeast, for example, Saccharomyces pastorianus W34/70; Saccharomyces carlsbergensis NCYC453 or NCYC456; or Saccharomyces cerevisiae NBRC1951, NBRC1952, NBRC1953 or NBRC1954, etc., may be used. Further, wine yeasts such as wine yeasts #1, 3 and 4 from the Brewing Society of Japan; and sake yeasts such as sake yeast #7 and 9 from the Brewing Society of Japan, baker's yeast such as NBRC0555, NBRC1346 and NBRC2043, etc., may also be used but not limited thereto. In the present invention, lager brewing yeasts such as Saccharomyces pastorianus may preferably be used. The reference yeast and the test yeasts may be selected from the above yeasts in any combination.

### EXAMPLES

Hereinafter, the present invention will be described in more detail with reference to working examples. The present invention, however, is not limited to the examples described below.

### Example 1

### Cloning of Gene Encoding Protein Having Trehalose Synthesis-promoting Activity (non-ScTSL1)

A gene encoding a protein having a trehalose synthesis-promoting activity of lager brewing yeast (non-ScTSL1) (SEQ ID NO: 1) was found as a result of a search utilizing the comparison database described in Japanese Patent Application Laid-Open No. 2004-283169. Based on the acquired nucleotide sequence information, primers non-ScTSL1_for (SEQ ID NO: 3) and non-ScTSL1_rv (SEQ ID NO: 4) were designed to amplify the full-length of the gene. PCR was carried out using chromosomal DNA of a genome sequencing strain, Saccharomyces pastorianus Weihenstephan 34/70 (sometimes abbreviated as “W34/70 strain”), as a template to obtain DNA fragments including the full-length gene of non-ScTSL1.

The non-ScTSL1 gene fragments thus obtained were inserted into pCR2.1-TOPO vector (Invitrogen) by TA cloning. The nucleotide sequences of the non-ScTSL1 gene were analyzed by Sanger's method (F. Sanger, Science, 214: 1215, 1981) to confirm the nucleotide sequence.

### Example 2

### Analysis of Expression of non-ScTSL1 Gene during Beer Fermentation

A beer fermentation test was conducted using a lager brewing yeast, Saccharomyces pastorianus W34/70, and mRNA extracted from the lager brewing yeast during fermentation was detected by a beer yeast DNA microarray.

| | |
|---|---|
| Wort extract concentration | 12.69% |
| Wort content | 70 L |
| Wort dissolved oxygen concentration | 8.6 ppm |
| Fermentation temperature | 15° C. |
| Yeast pitching rate | 12.8 × 10⁶ cells/mL |

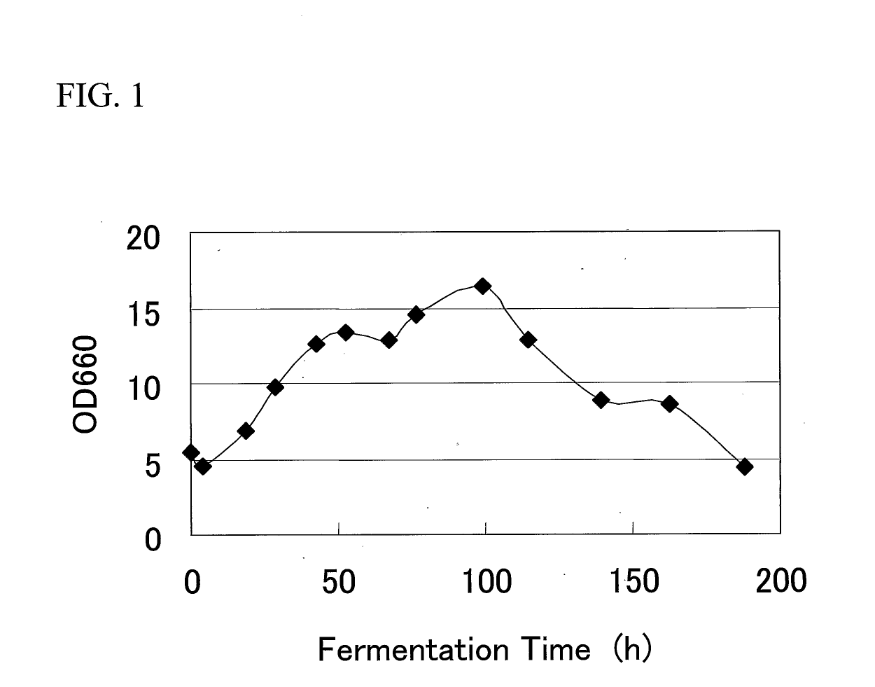

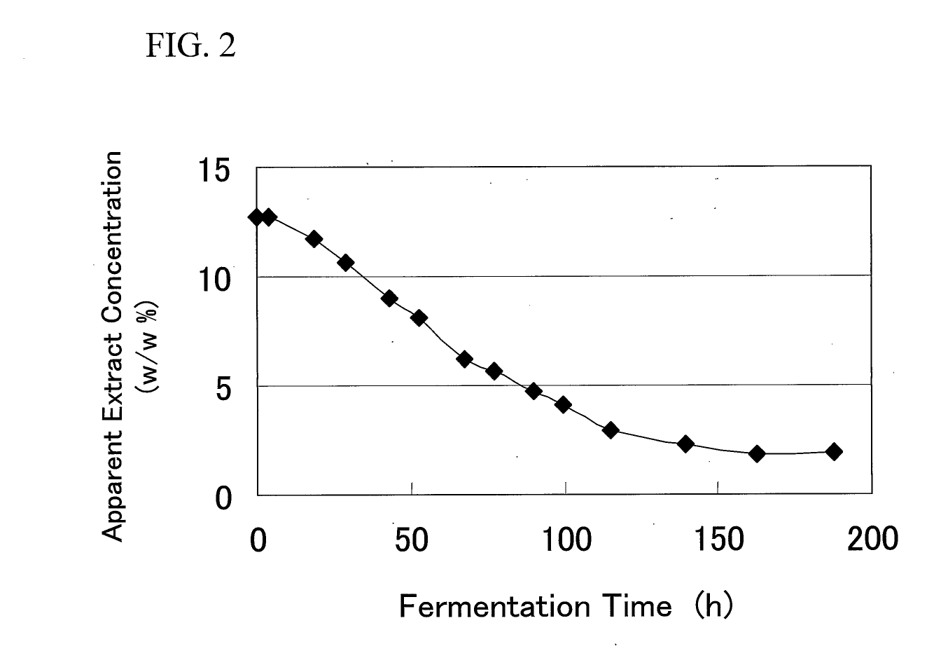

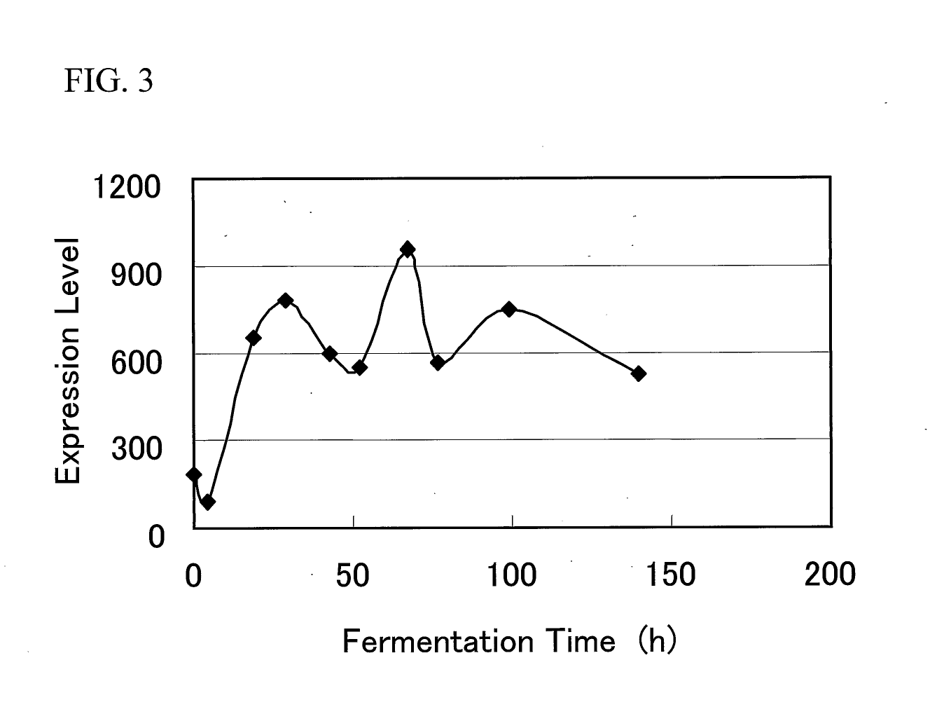

The fermentation liquor was sampled over time, and the time-course changes in amount of yeast cell growth (FIG. 1) and apparent extract concentration (FIG. 2) were observed. Simultaneously, yeast cells were sampled to prepare mRNA, and the prepared mRNA was labeled with biotin and was hybridized to a beer yeast DNA microarray. The signal was detected using GeneChip Operating system (GCOS; GeneChip Operating Software 1.0, manufactured by Affymetrix Co). Expression pattern of the non-ScTSL1 gene is shown in FIG. 3. This result confirmed the expression of the non-ScTSL1 gene in the general beer fermentation.

### Example 3

### Construction of non-ScTSL1 Highly Expressed Strain

The non-ScTSL1/pCR2.1-TOPO described in Example 1 was digested with the restriction enzymes SacI and NotI to prepare a DNA fragment containing the entire length of the protein-encoding region. This fragment was ligated to pYCGPYNot treated with the restriction enzymes SacI and NotI, thereby constructing the non-ScTSL1 high expression vector non-ScTSL1/pYCGPYNot. pYCGPYNot is a YCp-type yeast expression vector. A gene inserted is highly expressed by the pyruvate kinase gene PYK1 promoter. The geneticin-resistant gene G418^{r} is included as the selectable marker in the yeast, and the ampicillin-resistant gene Amp^{r} as the selectable marker in Escherichia coli.

Using the high expression vector prepared by the above method, an AJL4004 strain was transformed by the method described in Japanese Patent Application Laid-open No. H07-303475. The transformants were selected on a YPD plate medium (1% yeast extract, 2% polypeptone, 2% glucose and 2% agar) containing 300 mg/L of geneticin.

### Example 4

### Evaluation of Drying-Resistant Property of non-ScTSL1 Highly Expressed Strain

Drying-resistant properties of the parent strain (AJL4004 strain) and the non-ScTSL1 highly expressed strain obtained by the method described in Example 3 were evaluated by a method described below.

One platinum 1oopful of each yeast was inoculated into 10 mL of wort containing 100 mg/L of geneticin, and stirred at 30° C. overnight (precultivation). The precultivation liquid was inoculated into 10 mL wort containing 100 mg/l of geneticin to make its OD660=0.5, then main culture was initiated. The culture was continued for 2 days until the growth of the yeast reached stationary phase. Turbidity of the culture was measured at the completion of the culture, then the culture liquid was suspended in sterile water to make its OD=2. One hundred microliter (100 μL) of the suspension thus obtained was dispensed into a 1.5 mL microtube, then the yeast cells were dried by evaporation for 1 hour using a reduced-pressure concentrator (DNA110 SpeedVac (registered trademark), manufactured by ThermoSavant).

Viable cell ratio was measured by a method described below. The dried yeast cells obtained above were resuspended in 50 μL of sterile water, then 50 μL of 0.02% methylene blue solution (pH 4.5) was added to the suspension. Blue-stained yeast cells which had lost reducing power were considered as dead yeast cells. Then the suspension was observed under a microscope, and viable cell ratio was measured using a Cell Vital Analyzer System (DA cell counter, manufactured by Yamato Scientific Co., Ltd.). The cells were counted until the population reached more than 2000 cells to minimize experimental error.

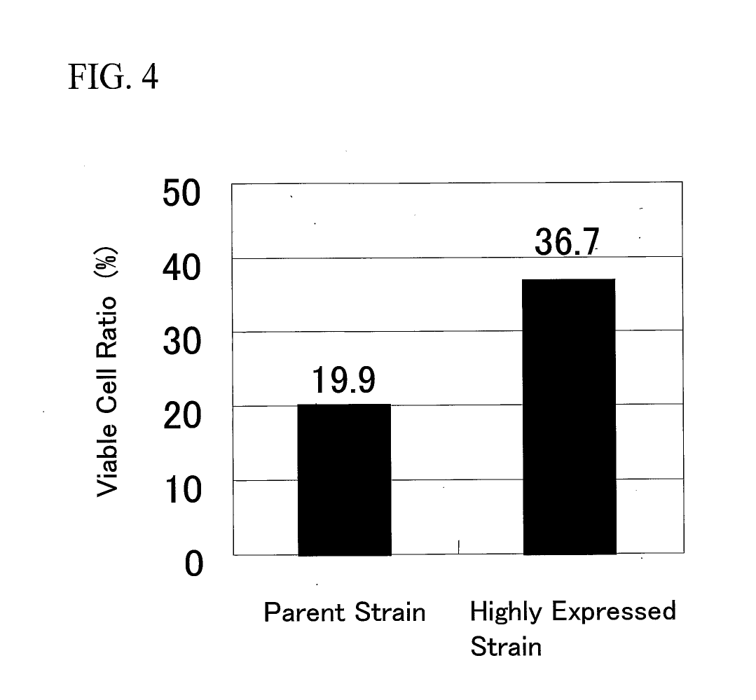

As indicated in FIG. 4, viable cell ratio of the highly-expressed strain was 36.7%, though viable cell ratio of the parent strain was 19.9%. It was demonstrated by the results that drying-resistant property of yeast was increased by high expression of non-ScTSL1.

### Example 5

### Evaluation of Low-temperature Resistant Property of non-ScTSL1 Highly Expressed Strain

Low-temperature resistant property of the parent strain (AJL4004 strain) and the non-ScTSL1 highly expressed strain obtained by the method described in Example 3 are evaluated by the method described below. Nine hundred microliter (900 μL) of the yeast suspensions cultured by the method described in Example 4 and prepared as OD660=2 are dispensed into two microtubes, respectively. One hundred microliter (100 μL) of sterile water is added to one of the microtubes, on the other hand, 100 μL of 99.5% ethanol is added to another one (final concentration is 10%). The suspensions are stored at 5° C. for 4 weeks, then viable cell ratios are measured by the same method as Example 4.

### INDUSTRIAL APPLICABILITY

According to the present invention, yeast can be stored stably for extended period of time, because drying-resistant property and/or low-temperature storage-resistant property can be enhanced by the present invention. Accordingly, efficiency of brewing alcoholic beverages (such as beer), production of bread, or manufacturing useful materials such as industrial alcohol production or production of useful proteins, etc. can be improved by the present invention.

1. A polynucleotide selected from the group consisting of:
(a) a polynucleotide comprising a polynucleotide consisting of the nucleotide sequence of SEQ ID NO:1;
(b) a polynucleotide comprising a polynucleotide encoding a protein consisting of the amino acid sequence of SEQ ID NO:2;
(c) a polynucleotide comprising a polynucleotide encoding a protein consisting of the amino acid sequence of SEQ ID NO:2 in which one or more amino acids thereof are deleted, substituted, inserted and/or added, and having a trehalose synthesis-promoting activity;
(d) a polynucleotide comprising a polynucleotide encoding a protein having an amino acid sequence having 60% or higher identity with the amino acid sequence of SEQ ID NO:2, and said protein having a trehalose synthesis-promoting activity;
(e) a polynucleotide comprising a polynucleotide which hybridizes to a polynucleotide consisting of a nucleotide sequence complementary to the nucleotide sequence of SEQ ID NO:1 under stringent conditions, and which encodes a protein having a trehalose synthesis-promoting activity; and
(f) a polynucleotide comprising a polynucleotide which hybridizes to a polynucleotide consisting of a nucleotide sequence complementary to the nucleotide sequence of the polynucleotide encoding the protein having the amino acid sequence of SEQ ID NO:2 under stringent conditions, and which encodes a protein having a trehalose synthesis-promoting activity.

2. The polynucleotide according to claim 1 selected from the group consisting of:
(g) a polynucleotide comprising a polynucleotide encoding a protein consisting of the amino acid sequence of SEQ ID NO: 2, or encoding the amino acid sequence of SEQ ID NO: 2 in which 1 to 10 amino acids thereof are deleted, substituted, inserted, and/or added, and wherein said protein has a trehalose synthesis-promoting activity;
(h) a polynucleotide comprising a polynucleotide encoding a protein having 90% or higher identity with the amino acid sequence of SEQ ID NO: 2, and having a trehalose synthesis-promoting activity; and
(i) a polynucleotide comprising a polynucleotide which hybridizes to a polynucleotide consisting of a nucleotide sequence of SEQ ID NO: 1 or which hybridizes to a polynucleotide consisting of a nucleotide sequence complementary to the nucleotide sequence of SEQ ID NO: 1, under high stringent conditions, which encodes a protein having a trehalose synthesis-promoting activity.

3. The polynucleotide according to claim 1 comprising a polynucleotide consisting of the nucleotide sequence of SEQ ID NO: 1.

4. The polynucleotide according to claim 1 comprising a polynucleotide encoding a protein consisting of the amino acid sequence of SEQ ID NO: 2.

5. The polynucleotide according to claim 1, wherein the polynucleotide is DNA.

6. A protein encoded by the polynucleotide according to claim 1.

7. A vector containing the polynucleotide according to claim 1.

8. A yeast into which the vector according to claim 7 has been introduced.

9. The yeast according to claim 8, wherein drying-resistant property is increased.

10. The yeast according to claim 8, wherein low-temperature storage-resistant property is increased.

11. The yeast according to claim 9, wherein the drying-resistant property is increased by increasing an expression level of the protein encoded by the polynucleotide.

12. The yeast according to claim 10, wherein the low-temperature storage-resistant property is increased by increasing an expression level of the protein encoded by the polynucleotide.

13. A method for producing an alcoholic beverage by using the yeast according to claim 8.

14. The method according to claim 13, wherein the brewed alcoholic beverage is a malt beverage.

15. The method according to claim 13, wherein the brewed alcoholic beverage is wine.

16. An alcoholic beverage produced by the method according to claim 13.

17. A method for assessing a test yeast for its drying-resistant property and/or low-temperature storage-resistant property, comprising using a primer or probe designed based on the nucleotide sequence of a gene having the nucleotide sequence of SEQ ID NO: 1 and encoding a trehalose synthesis-promoting activity.

18. A method for assessing a test yeast for its drying-resistant property and/or low-temperature storage-resistant property, comprising: culturing the test yeast; and measuring the expression level of the gene having the nucleotide sequence of SEQ ID NO: 1 and encoding a trehalose synthesis-promoting activity.

19. A method for selecting a yeast, comprising: culturing test yeasts; quantifying the protein of claim 6 or measuring the expression level of the gene having the nucleotide sequence of SEQ ID NO: 1 and encoding a protein having a trehalose synthesis-promoting activity; and selecting a test yeast having an amount of the protein or the gene expression level according to favorable drying-resistant property and/or low-temperature storage-resistant property.

20. The method for selecting a yeast according to claim 19, comprising: culturing a reference yeast and test yeasts; measuring for each yeast the expression level of the gene having the nucleotide sequence of SEQ ID NO: 1 and encoding a trehalose synthesis-promoting activity; and selecting a test yeast having the gene expression higher than that in the reference yeast.

21. The method for selecting a yeast according to claim 19, comprising: culturing a reference yeast and test yeasts; quantifying the protein encoded by the polynucleotide in each yeast; and selecting a test yeast having a larger amount of the protein than that in the reference yeast.

22. A method for producing an alcoholic beverage comprising: conducting fermentation using the yeast according to claim 8 or a yeast selected by the methods according to claim 19.


SEQUENCE LISTING (4 sequences)

SEQ ID NO: 1 - DNA, 3309 bp, Saccharomyces sp.
atggctctcatcgtggcatcgttgtttttgccgtaccaaccgcaattcgaactagacacctctctacccgagaactcacaggtggacccgtccctggtgaacgttcactccaagggaagcgaccagcagcaccgcgcgctatcgaacaaccactcgcaagagtcgttggtcgcgcccgctcctgagcagggcgtgcccccagcaatctccaggagcgccaccaggtcgcctatatctttcaatcgcgcctcgtccaccaacacggccaacctggacgatctcgtttcctcggacgtgtttctggagaatcttaccgccaacgccaccacgtcgcacacgcccacgagcaagacgatgctcaagccgcgcaacaacggctccgtggagcagttcttctcgtcctcctccaacgtcccctcggaccgcattgcgtcgcccatccaattccagcaggactccggctcgaggatcgcgtcgccaatccagcagcaggaccccacagccaacctgctgaagaacgtgaacaagtcgctgctagtgcactcgctgctgaacaacacctcgcagaccagcctcgacaagccgcacaatcacatcgtgactccgaagtcccgggcgggcaacaagtccgcctccgcggcttcctctctggtaaacaaggccaaacaggcgcccgcctcggcttcctcttcttcctcctccgctgctccgccctctatcaagcggatttccccgcatttagcggccgcagcggccgctgccgctgcgaagcaacggcccatcctggccaagcaaccgtccaacctcaagtactcggagctagcagacatctcgtccagtgagacgtcctcgcagcacaacgagtctgacccggaggagctcacgggcgtgcccgacgaggagtacgtctcggacctggaaatggatgacgccaagcaggattacaaggttccgaagttcggcggctattccaacaagtcacagctcaagaaatactcgcttttaaggtcgacgcaagagctgttcagccgtctgccttggtccattgttccctctatcaaagggaacggtgccatgaagaacgccatcaacacagccgtcttggagaacatcatcgcgcaccaccacgtcaagtgggtcgggaccgtgggcatccccacggacgaggtcccggagaacatccttggcaaaatctccgactcgctgagggacgattacgactcttattccgttctcacggacgacgtcacgttcaaagccgcatacaagaactactgtaaacaaatcttgtggcccactctgcactaccagatcccggacaacccgaactccaaggccttcgaggatcactcctggaagttctacaagcacatgaaccagcagttcgcggacgcgatcgtgaagatatacaaggagggtgacaccatctgggtccatgactaccatctgatgctggtcccgcagatgattagagacgtcttgccctccgccaagatcgggttcacgctgcacgtctcgttccccagtagcgaggtgttcaggtgtctggcccagagggagaagatcttggagggactcactggtgcggatttcgttgggttccagaccaaagaatacgcgagacacttcttgcagacgtccaaccgattgctgatggcggatgtggtgcatgacgaagagttgaagtacaacggccgggtcgtgtccgtgaagttcactcccgtcggcatagacgccttcgacctccaatcgcaactgaaggacgaacacgtcattcaatggcgccacctgatccgtgaaagatggcagaacaagaaactgattgtttgtcgtgaccagttcgacagaatcaggggcatccataaaaaactgctggcgtacgaaaaattcttggcggacaacccacagtacgtggagcagctgaccatgatccagatctgtatcggcaacagcaaagacgtcgaactggagcgccagatcatgctcgtcgtagacagaatcaactcgctgtccacgaacatcagcatttcgcagcctgtagtgttcttgcatcaggacctggacttctctcagtacctcgcattgagttcggaagccgacctgttcgtggtcagctccctgagggaaggtatgaatttgacctgccacgaatacattgtctgctccgaggacaagaattccgcgctgttgctgtctgaattcaccggcagcgcctcgctgttgaacgatggcgctatattaatcaatccatgggatacaaagaactttgcttcgtctatccgcaaaggtttggagatgccatttgatgaaagaagaccgcagtggaagaaactaatgaaggacatcatcaacaatgactccaccaactggatcaagtcctctctgcaagatatccacttttcgtggaagttcaaccaagagggttccaagatcttcaaattgaacacgaaaaacctgtcagatgattatcagtcttccaagaaacgcatgttcgtctttaacatcgctgaacctccgacttcgagaatgatttccatactgaacgatatgacgtcgaagggcaacatcgtgtacatcatgaactcgtttccaaaggcgattctagaaaacctctacagtcgtgtgcaaaaccttgggctgatcgcagaaaacggtgcctacgtaagtctgaatggtgtgtggtacaacatcgtggaccaaatcgactggcgtaacgacgtggccaagatcctgaaggacaaagtggaaagattgcccggttcatactataagatcaacgactcgatgatcaagttccacaccgagaacgcagaggaccaagaccgtgttgccagcgtcattggtgaagccatcacgcacatcaacaccgttttcgaccacaggggtatccacgcttatgtttacaaaaacgtcgtctcggtacagcaaactggtctttccttgtctgcagcccaattccttttcaggttctacaactccgcctcggatccactagacacaagttcgggccagatcacgaatatccattcgccatcacattcgcaatcggactccctagatcaagaacaacaagcaccaccagcttcccccacggtgtcgctaaaccacattgattttgcatgtgtatccggttcgtcgtctcccgtgttggaaccattgttcaagctggtcaatgacgaagctagcgatggtcaagtgaaggtcggccatgccatcgtttacggcgatgccacttccacttatgccaaggaacacgtaaatgggctaaacgaacttttcacgattttttctagaatcatcgaaaattga

SEQ ID NO: 2 - protein, 1102 aa, Saccharomyces sp.
MALIVASLFLPYQPQFELDTSLPENSQVDPSLVNVHSKGSDQQHRALSNNHSQESLVAPAPEQGVPPAISRSATRSPISFNRASSTNTANLDDLVSSDVFLENLTANATTSHTPTSKTMLKPRNNGSVEQFFSSSSNVPSDRIASPIQFQQDSGSRIASPIQQQDPTANLLKNVNKSLLVHSLLNNTSQTSLDKPHNHIVTPKSRAGNKSASAASSLVNKAKQAPASASSSSSSAAPPSIKRISPHLAAAAAAAAAKQRPILAKQPSNLKYSELADISSSETSSQHNESDPEELTGVPDEEYVSDLEMDDAKQDYKVPKFGGYSNKSQLKKYSLLRSTQELFSRLPWSIVPSIKGNGAMKNAINTAVLENIIAHHHVKWVGTVGIPTDEVPENILGKISDSLRDDYDSYSVLTDDVTFKAAYKNYCKQILWPTLHYQIPDNPNSKAFEDHSWKFYKHMNQQFADAIVKIYKEGDTIWVHDYHLMLVPQMIRDVLPSAKIGFTLHVSFPSSEVFRCLAQREKILEGLTGADFVGFQTKEYARHFLQTSNRLLMADVVHDEELKYNGRVVSVKFTPVGIDAFDLQSQLKDEHVIQWRHLIRERWQNKKLIVCRDQFDRIRGIHKKLLAYEKFLADNPQYVEQLTMIQICIGNSKDVELERQIMLVVDRINSLSTNISISQPVVFLHQDLDFSQYLALSSEADLFVVSSLREGMNLTCHEYIVCSEDKNSALLLSEFTGSASLLNDGAILINPWDTKNFASSIRKGLEMPFDERRPQWKKLMKDIINNDSTNWIKSSLQDIHFSWKFNQEGSKIFKLNTKNLSDDYQSSKKRMFVFNIAEPPTSRMISILNDMTSKGNIVYIMNSFPKAILENLYSRVQNLGLIAENGAYVSLNGVWYNIVDQIDWRNDVAKILKDKVERLPGSYYKINDSMIKFHTENAEDQDRVASVIGEAITHINTVFDHRGIHAYVYKNVVSVQQTGLSLSAAQFLFRFYNSASDPLDTSSGQITNIHSPSHSQSDSLDQEQQAPPASPTVSLNHIDFACVSGSSSPVLEPLFKLVNDEASDGQVKVGHAIVYGDATSTYAKEHVNGLNELFTIFSRIIEN

SEQ ID NO: 3 - DNA, 40 bp, Artificial
  Primer
gagctcatagcggccatggctctcatcgtggcatcgttgt

SEQ ID NO: 4 - DNA, 42 bp, Artificial
  Primer
ggatcctatgcggccgcataatttgttcattaaagagagtag